(19) United States
(12) Patent Application Publication
(10) Pub. No.: US 2002/0151875 A1
(43) Pub. Date: Oct. 17, 2002

(54) Patient activated administration of drug bolus from implantable drug delivery system

(76) Inventor: Markus Haller, Penthalaz (CH)

Correspondence Address: Medtronic, Inc., 710 Medtronic Parkway, Minneapolis, MN, 55432, US

(21) Appl. No.: 10/004,848

(22) Filed: Dec. 4, 2001

Related U.S. Application Data
(60) Provisional application No. 60/251,214, filed on Dec. 4, 2000.

(51) Int. Cl. A61K 9/22

(52) U.S. Cl. 604/891.1; 604/67

(57) ABSTRACT

Systems and methods are described for reducing the risk of overdosage from a patient activated implantable drug delivery system. In one embodiment, an implantable drug delivery system is configured to deliver a drug. An external activation unit is operable by a user to request activation of the implantable drug delivery system. The activation unit includes a controller to reject a request to activate the implantable drug delivery system prior to expiration of a lockout interval. The controller issues an activation signal to the implantable drug delivery system in response to a request received after expiration of the lockout interval. The controller restarts the lockout interval upon activating the drug delivery system.

This Application claims Priority From U.S. Provisional Application No. 60/251,214, Filed Dec. 4, 2000, The Entire Content Of Which Is Incorporated Herein By Reference.

### FIELD OF THE INVENTION

The invention relates to implantable drug delivery systems for delivering pharmaceutical agents or other fluids to a desired location in a body of a patient.

### BACKGROUND

Pharmaceutical agents and other fluids are increasingly being administered to patients through the use of drug delivery systems. Some of these drug delivery systems are designed to be mounted externally to the body, and are connected to a catheter introduced to the body of the patient. Other systems have comprised an implantable pump that is mounted subcutaneously within the body of the patient, and which delivers a drug to the body at a desired location.

An example type of implantable drug delivery system is an infusion pump. The device typically includes a catheter and a pump section having a collapsible reservoir and a fill port for refilling the reservoir with fresh drug preparation. The infusion pump may automatically deliver a controlled amount of medication through the catheter using an electronically driven pump. The dosage, rate, and timing can be programmed into electronics contained within the pump from an external programming device. The external programmer typically transmits programming information using radio waves.

Many of the external drug delivery systems offer a mechanism by which a user, such as a patient, can request the delivery of a drug bolus. This feature is often referred to as “on-demand administration” of a drug. When the drug delivery system is provided for pain control, the on-demand feature can help the patient cope with variations in the level of pain. External drug delivery systems typically include a button or other simple interface by which the user can request delivery of the drug.

One problem with conventional implantable drug delivery systems is the failure to offer a convenient and easy to use feature in which the patient can request administration of the drug. With the lack of such a feature, the patient or physician may not be able to quickly address variations in the level of pain, as can be done with external drug delivery systems.

Another problem with conventional drug delivery systems and on-demand administration of drugs is the failure to address various problems related to excessive requests for the drug by the patient. In particular, the conventional drug delivery systems often fail to adequately address the possibility of overdosage by the patient. A number of the external drug delivery systems, for example, include a hardwired limit for the dispensing of medication. If requests exceed the defined limits, the external system will inhibit the delivery of excessive medication to the patient.

Other external drug delivery systems seek to prevent patient overdose by maintaining a count of a dispensed drug, and comparing the count to a number of medication requests. The external drug delivery system correlates the number of medication requests with actual medication dispensed to verify proper operation. Still other external drug delivery systems seek to prevent overdosage by storing pills that were not taken on time so that such medications cannot be taken or otherwise improperly used by the patient.

Among other limitations, these approaches fail to address the potential for the patient to request numerous dosages within a short time span. Examples of conventional techniques and/or devices may be found in the issued U.S. Patents listed in Table 1 below.

TABLE 1

| U.S. Pat. No. | Inventor | Issue Date |
|---|---|---|
| 4,619,653 | Fischell | Oct. 28, 1986 |
| 5,392,952 | Bowden | Feb. 28, 1995 |
| 5,507,277 | Rubsamen et al | Apr. 16, 1996 |
| 5,871,478 | Berrigan | Feb. 16, 1999 |
| 4,627,839 | Young | Dec. 9, 1986 |

All patents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY OF THE INVENTION

The present invention has certain objects. That is, various embodiments of the present invention provide solutions to one or more problems existing in the prior art with respect to implantable drug delivery devices or systems. These problems include, for example, the lack of a provision in the prior art for a convenient and easy to use mechanism for a patent to activate an implantable drug delivery system, thereby allowing the patient to request administration of the drug as needed. Other problems include the lack of adequate safety mechanisms to prevent overdosage to a patient having an implanted drug delivery system. Various embodiments of the present invention have the object of solving at least one of the foregoing problems.

It is, therefore, an object of the invention to provide a convenient and easy to use mechanism by which a patient can request administration of the drug from an implanted drug delivery system. By providing such a mechanism, the patient has at least some control over his or her therapy, and can more readily treat pain and customize the therapy as needed.

It is a further object of the invention to provide safety mechanisms to prevent overdosage by a patient having an implanted drug delivery device. By providing such safety mechanisms, risk of overdosage is minimized, yet the patient has the benefit of being able to activate the implanted drug delivery system as needed.

It is a further object of the invention to provide an implanted drug delivery device having a lockout mechanism by which requests for medication during a lockout interval are rejected. It is a further object of the invention to provide an implanted drug delivery device having a lockout mechanism responsive to a programmable lockout interval.

It is a further object of the invention to provide an activation device for initiating delivery of medication from an implanted drug delivery device in response to input from a user, and for indicating the success or failure of the requests.

Various embodiments of the invention may possess one or more features capable of fulfilling the above objects. In general, the invention is directed to an implantable drug delivery system and an external activation unit for activating the drug delivery system. The activation unit contains a safe lockout mechanism that controls the rate at which the patient can request additional boluses of the drug. Alternatively, the lockout mechanism may control the rate at which the patient may trigger a drug bolus incremental to a pre-programmed drug schedule. The invention, however, is not so limited as to the implementation of the lockout mechanism within the activation unit. These and other safety features may readily be incorporated directly into drug delivery system.

The invention is directed, in one embodiment, to a method of activating a drug delivery system including maintaining a timer to time a lockout interval, and rejecting a user request to activate the implantable drug delivery system prior to expiration of the lockout interval. The implantable drug delivery system is activated in response to a user request received after expiration of the lockout interval, and the lockout interval is restarted.

The invention may also be embodied as an apparatus comprising an input/output (I/O) device and a controller coupled to the I/O device to receive a user request to activate an implantable drug delivery system. The apparatus also comprises a timer managed by the controller to time a lockout interval. The controller outputs an activation signal to activate the implantable drug delivery system when the user request is received after expiration of the lockout interval. The apparatus may comprise and external activation device and may further include a telemetry unit to transmit the activation signal to the implantable drug delivery system. Alternatively, the apparatus may comprise the drug delivery system. In this embodiment, the I/O device may comprise a telemetry unit to receive a telemetric communication conveying the user request.

The invention may also be embodied as a system comprising an implantable drug delivery system, and an external activation unit operable by a user to request activation of the implantable drug delivery system. The activation unit may include a controller to reject a user request to activate the implantable drug delivery system prior to expiration of a lockout interval.

The invention offers one or more advantages. For example, by providing a mechanism for requesting administration of drug from an implanted drug delivery system, the patient has at least some control over his or her therapy, and can more readily treat pain and customize the therapy as needed. Furthermore, by making use of a lockout interval, the risk of overdosage by the patient is reduced. In addition, the lockout mechanism may be responsive to a programmable lockout interval, allowing a clinician the flexibility to control the lockout interval based on the particular circumstances and needs of the patient.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic illustration of an exemplary system having a drug delivery system implanted in a patient and in communication with an exemplary external activation device.

FIG. 2 is a timing diagram that illustrates operation of the exemplary external activation device that reduces the risk of overdosage by the patient by making use of a lockout interval.

FIG. 3 is a schematic front view of an example embodiment of the external activation device of FIG. 1.

FIG. 4 is a schematic front view of another example embodiment of the external activation device of FIG. 1.

FIG. 5 is a block diagram illustrating the constituent components of an example embodiment of the external activation device of FIG. 1.

FIG. 6 is a block diagram illustrating the constituent components of an example embodiment of the implantable medical device of FIG. 1.

FIGS. 7 and 8 illustrate a flow diagram of the operation of the system and components of FIG. 1.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is to be understood that other embodiments may be utilized and structural or logical changes may be made without departing from the scope of the present invention. The following detailed description, therefore, is not to be taken in a limiting sense, and the scope of the present invention is defined by the appended claims.

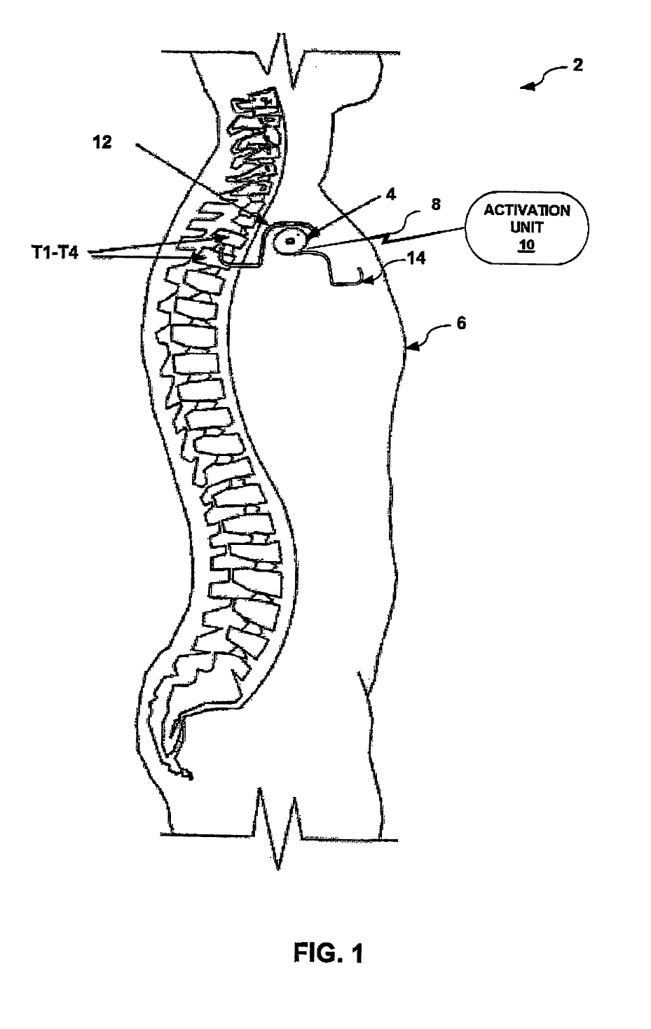

FIG. 1 is a schematic illustration of an exemplary system 2 having a drug delivery system 4 implanted in a patient 6 and in communication with an exemplary external activation device 10. Drug delivery system 4 may include one or more drug pumps that are operable to pump a programmed bolus of a drug from a drug reservoir through a drug delivery catheter 12 to a location within patient 6, such as spinal segments T1-T4. Drug delivery catheter 12 may be a Medtronic Model 8700 series catheter. Drug delivery system 4 typically includes an embedded controller or microprocessor to control the timing of delivery and amount of drug delivered. Drug delivery system 4 may be percutaneously refillable through a septum 14, or may include a self-sealing reservoir that may be refilled by a needle and syringe.

External activation unit 10 is in communication with drug delivery system 4 via communication link 8, and provides a mechanism by which a user, such as patient 6 or a physician, can activate drug delivery system 4. Communication link 8 may comprise any one of a variety of communications including radio frequency (RF) transmissions, magnetic fields, and the like.

Upon receiving input from the user, activation unit 10 may output an activation signal via link 8. Drug delivery system 4 may respond to the activation signal in a number of ways. In one embodiment, for example, drug delivery system 4 administers a bolus of a drug to patient 6 via catheter 12 in response. In this manner, system 2 provides a convenient and easy to use feature by which patient 6 can request administration of a bolus by drug delivery system 4. In addition, drug delivery system may increase an amount of drug to deliver per bolus. Advantageously, the patient has at least some control over his or her therapy, and can more readily treat pain and customize the therapy as needed.

In another embodiment, drug delivery system 4 operates in a continuous mode in which a stream of drug is pumped to the patient 6 according to a pre-programmed drug schedule. In this embodiment, drug delivery system 4 may adjust a flow rate of the drug in response to the activation signal. Drug delivery system 4 may, for example, increase or decrease the flow rate of the drug based upon data communicated from activation unit 10. In other words, the patient may trigger a drug bolus increment into the pre-programmed drug schedule.

As described in further detail below, activation unit 10 includes features that address various problems related to excessive requests for drug by the patient. In particular, the activation unit 10 maintains a lockout interval that can be programmatically set by a clinician. Upon successfully issuing an activation request to drug delivery system 4, activation unit 10 initiates a lockout interval during which further user requests are rejected. In other words, the user can engage activation unit 10 to direct drug delivery system 4 to deliver a first bolus of the drug, for example, but is unable to request an additional bolus until the lockout interval has expired.

In addition, activation unit 10 may determine whether the request would cause thresholds to be exceeded, as programmatically specified by the clinician. For example, activation unit 10 may determine whether delivery of an additional bolus would exceed a threshold number of dosages over a period of time. As another example, activation unit 10 may determine whether an adjustment to the flow rate may cause the flow rate to exceed a specified maximum flow rate, or fall below a minimum flow rate. In this manner, activation unit 10 provides a convenient mechanism for the user to activate drug delivery system 4, but provides safety features for reducing the risk of overdosage of patient 6.

For exemplary purposes, many of these features are described in reference to activation unit 10. However, the invention is not so limited as the use of a lockout interval and other safety features may readily be incorporated directly into drug delivery system 4. In this configuration, activation unit 10 may simply issue an activation request via communication link in response to input from the user without regard to the lock interval or other safety feature.

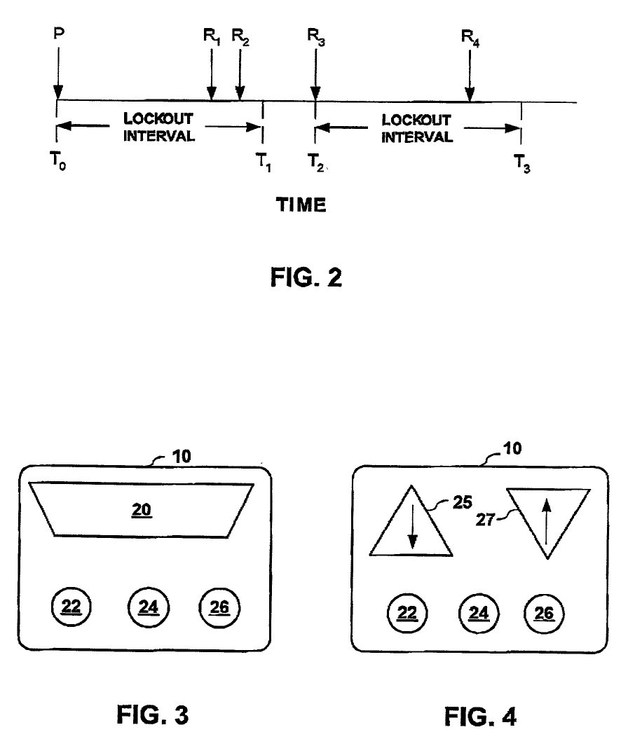

FIG. 2 is a timing diagram that further illustrates operation of the exemplary external activation device that reduces the risk of overdosage by the patient by making use of a lockout interval. Initially, activation unit 10 receives programmatic input P from a clinician at a time T₀. The clinician may, for example, programmatically set a lockout interval. In addition, the clinician may refill the drug reservoir of drug delivery system 4.

Upon receiving the programmatic input, activation unit 10 initiates a lockout 10 interval during which user requests R₁ and R₂ are rejected. In other words, activation unit 10 does not issue an activation signal to drug delivery system 4 via communication link 8 in response to user request R₁ and R₂ during the lockout interval.

Upon expiration of the lockout interval at time T₁, activation unit 10 no longer rejects user requests. In particular, activation unit 10 issues an activation signal to drug delivery system 4 in response to user request R₃ received at time T₂. Upon issuing the activation signal, activation unit restarts the lockout interval. Consequently, activation unit 10 rejects any user requests from time T₂ to time T₃, such as request R_{4.}

FIG. 3 is a schematic front view of an example embodiment of an external activation device 10A. In particular, activation unit 10A includes an activation button 20 and light emitting diodes (LEDs) 22, 24 and 26. This embodiment may be particularly useful for an embodiment in which implantable drug dispensing system 10 delivers a bolus of a drug in response to a user request. The user, for example, can easily issue a request for the administration of a drug bolus by pressing activation button 20. As described above, activation unit 10 may issue an activation signal to drug delivery system 4 in response to the user request based upon the lockout interval and other safety features. Upon issuing the activation signal, drug delivery system 4 may issue a response signal to activation unit 10 indicating whether the drug bolus was successfully delivered.

Activation unit 10A illuminates LEDs 22-26 to provide feedback to the user.

Activation unit 10A may, for example, illuminate LED 22 upon successfully activating drug delivery system 4 for administration of a bolus. Similarly, activation unit 10A may illuminate LED 24 if the user actuates activation button 20 during the lockout interval. In this manner, activation unit 10A informs the user that the request was rejected. Finally, activation unit 1OA may illuminate LED 26 to indicate that activation unit 10A was not able to establish communication with drug delivery system 4. Accordingly, in one embodiment, LEDS 22, 24, and 26 may be green, yellow and red, respectively.

FIG. 4 is a schematic front view of another example embodiment of an external activation device 10B. In particular, activation unit 10B includes an increase button 25, decrease button 27 and LEDs 22, 24 and 26. This embodiment may be particularly useful for an embodiment in which implantable drug dispensing system 10 continuously delivers a drug over a period of time. Using activation unit 10B, the user can easily issue requests to adjust the flow rate of the drug within programmatically defined limits. The user may, for example, request an increase in the flow rate by actuating button 25. Similarly, the user may request a decrease in the flow rate by actuating button 27. As described above, activation unit 10 may issue an activation signal to drug delivery system 4 in response to the user requests based upon the lockout interval and other safety features. Activation unit 10B illuminates LEDs 22, 24 and 26 to provide feedback to the user.

Although illustrated separately for purposes of example, these embodiments of activation unit 10 may readily be combined and adapted. An activatio n unit 10 may, for example, readily support both modes of operation. Furthermore, activation 10A may produce one or more audible sounds in response to user activation requests.

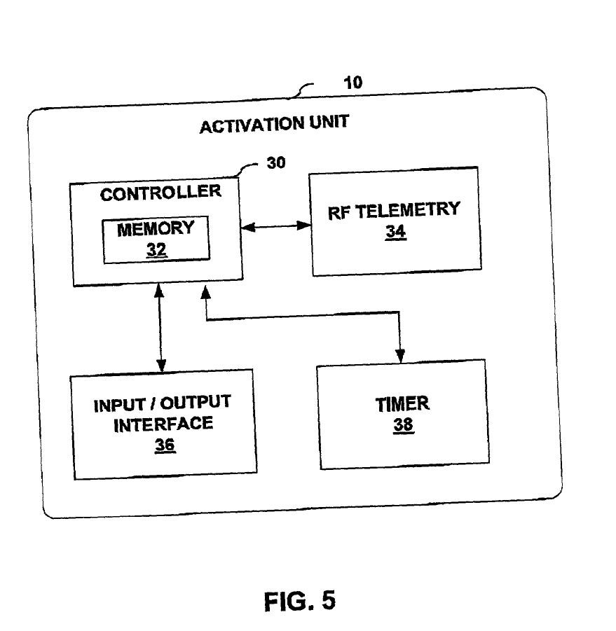

FIG. 5 is a block diagram illustrating the constituent components of an example embodiment of the external activation unit 10 of FIG. 1. In particular, activation unit includes input/output (I/O) interface 36 to receive user requests and to provide feedback to the user upon receiving such requests. I/O interface 36 may include, as described above, one or more buttons, LEDs and tone generators.

Controller 30 receives a request signal from I/O interface 36 in response to user input. Upon receiving the request signal, controller 30 determines whether to issue a request signal to drug delivery system 4 via radio frequency (RF) telemetry 34. In particular, the controller 30 controls timer 38 to time a lockout interval. Specifically, controller 30 initializes timer 30 to count up to, or count down from, a lockout interval. The lockout interval may be programmatically defined by a clinician via I/O interface 36. Alternatively, controller 30 may initialize timer 38 to a predefined lockout interval. Timer 38 may be a hardware-based timer, such as a timer commonly provided by a real-time clock, or may be a software-based timer.

RF telemetry 34 provides a mechanism for establishing RF communications between activation unit 10 and drug delivery system 4. Specifically, controller 30 engages RF telemetry 34 to issue activation signals to drug delivery system 4. Activation unit 10 may also receive response signals from drug delivery system 4 via RF telemetry 34.

Controller 30 includes memory 32 to store executable instructions and data.

Controller 30 may store, for example, the lockout interval, safety thresholds and other programmable data.

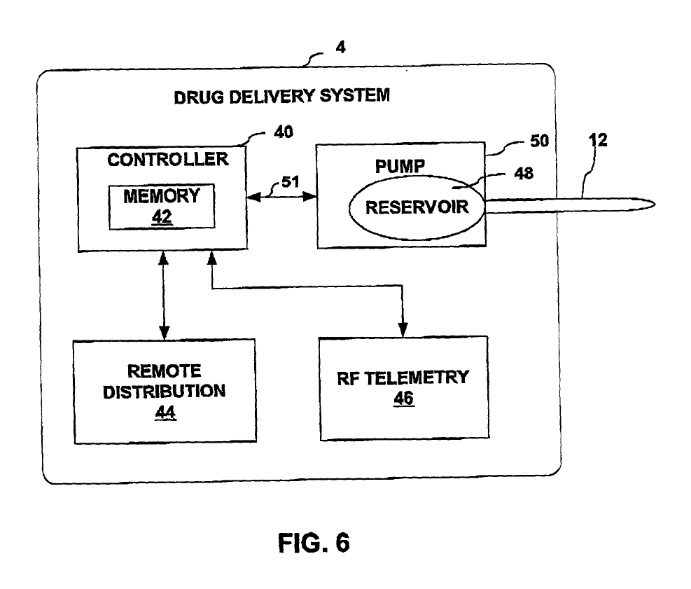

FIG. 6 is a block diagram illustrating the constituent components of an example embodiment of the implantable drug delivery system of FIG. 1. Controller 40 receives activation signals via RF telemetry 46, which communicates with activation unit 10 via communications link 8. Upon receiving an activation signal, controller 40 may output control 51 directing pump 50 to delivery a drug bolus. In particular, pump 50 includes reservoir 48, which may hold one of a wide variety of therapeutic drugs selected by a clinician based upon the particular needs of patient 6. Pump 50 dispenses the drug bolus from reservoir 48 to the body of patient 6 via catheter 12.

Examples of implantable pumps include a number of SynchroMed™pumps manufactured by and commercially available from Medtronic Inc. Pumps of this kind typically include self-sealing reservoirs that may be refilled by a needle and syringe, and need not be surgically removed when empty. The needle and syringe may also be used to drain a pump of one drug, flush the reservoir, and refilled the reservoir with a different drug. The pumps may further include a fill port (not shown in FIG. 6) that assists the medical personnel refilling the reservoir. The invention is not limited to use with SynchroMed pumps, however, and may be adapted for use with other models of implantable drug pumps.

Infusion apparatus, such as catheter 12, infuse drugs from reservoir 48 to one or more infusion sites the body of patient 6. The infusion site, and the drug being infused, may be selected based upon the needs of patient 6. For example, a catheter may deliver drugs to the patient's subclavian vein, or to the patient's SVC or to the patient's fatty tissue. If drug delivery system 4 has more than one catheter, the catheters need not deliver drugs to the same infusion site.

Controller 40 regulates the operation of drug delivery system 4 and may include memory 42. Memory 42 may be used to store therapy data, such as the amount of drug dispensed to the patient, the estimated amount of drug remaining in reservoir 48, the number of dosages supplied, therapy trends, and so forth.

A clinician may access this data by input/output devices such as remote distribution link 44 or RF telemetry 46. Remote distribution link 44 provides a channel for downloading data from patient 6 over a telephone line or over the Internet, for example. RF telemetry 46 provides immediate access to the data on a dedicated channel. Typically, a patient is required to visit the physician's office when data is to be downloaded via RF telemetry 46.

In this manner, input/output devices 44 and 46 allow a clinician, such as a physician, to exchange information with controller 40. The information exchanged may include drug delivery data, patient activity data, and other numbers, statistics or data.

Input/output devices 44 and 46 may also be used to program controller 40.

Controller 40 may access memory 42 to store the instructions or parameters programmed by the clinician and to retrieve stored instructions or parameters. The clinician may program, for example, minimum or maximum dosages, frequency of administration, and various other criteria for delivery of drugs.

Controller 40 may be housed inside drug delivery system 4 or may be a component separate from drug delivery system 4. Signals, instructions and data may be transmitted the components of drug delivery system 4 by hard wire, optical cable, wireless telemetry, and the like, or any combination thereof.

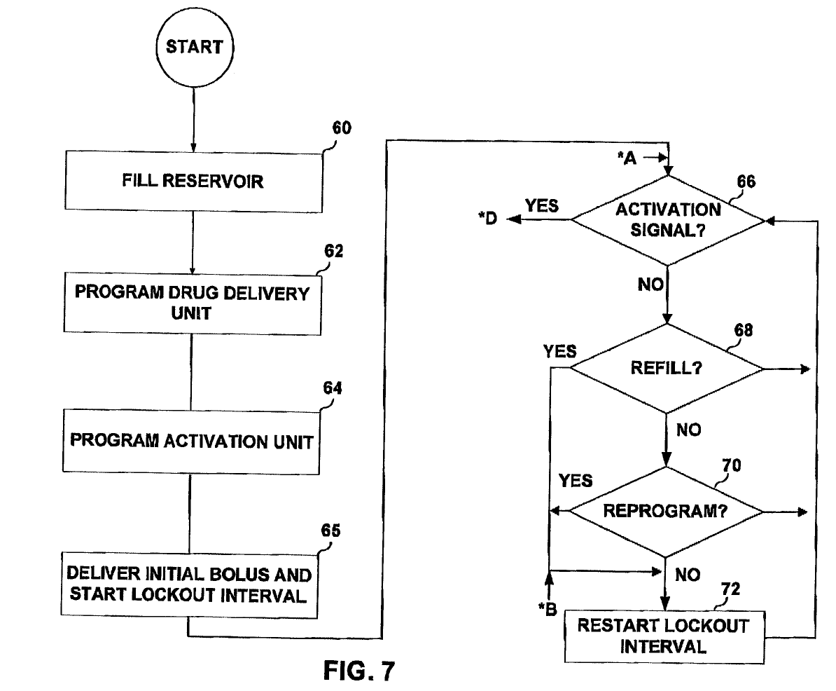

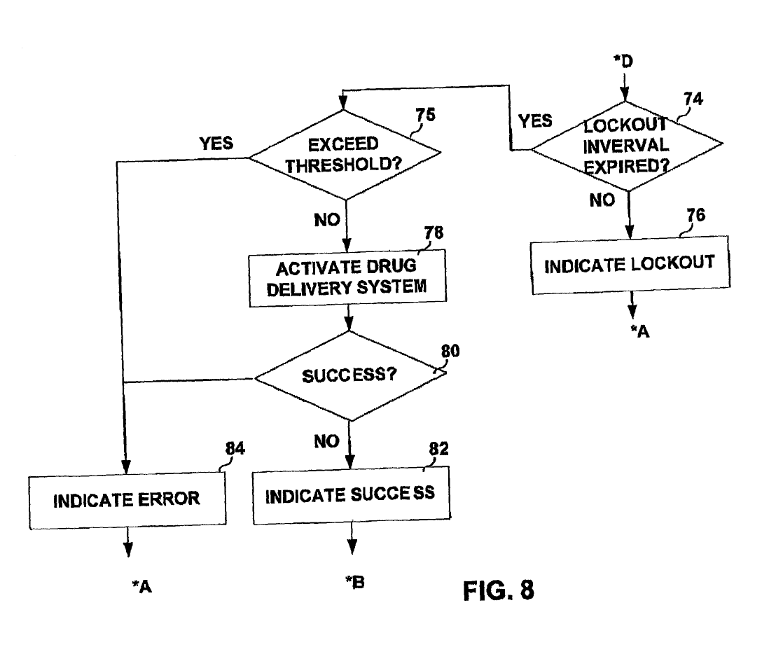

FIGS. 7 and 8 illustrate a flow diagram of one example mode of operation of the system and components of FIG. 1. Initially, a clinician fills reservoir 48 with a drug (60) and programs the drug delivery system 4 via remote distribution link 44 or RF telemetry 46 (62). As described above, the clinician may program, for example, minimum or maximum dosages, frequency of administration, and various other criteria for delivery of drugs.

Next, the clinician programs activation unit 10 (64). The clinician may programmatically set a lockout interval. In addition, the clinician may set one or more thresholds, such as a maximum number of dosages to be delivered over a period of time, or maximum and minimum flow rates. After programming, activation unit 10 may initiate a first lockout interval (65).

Next, activation unit 10 continually determines whether the user has requested the delivery of a drug bolus, i.e., the activation of drug delivery system 10 (66). This may take the form of polling I/O interface 36 or by way of interrupt-driven software. If the user has not requested the activation of drug delivery system 4, activation unit 10 determines whether the clinician has refilled reservoir 48 (68), or has reprogrammed activation unit 10 (70). If either event has occurred, activation unit 10 restarts the lockout interval (72).

If the user has actuated I/O interface 36 to request activation of drug delivery system 4 (yes branch of 66), activation unit 10 determines whether the lockout interval has expired (74 of FIG. 8). If not, activation unit 10 indicates that the user has tried to activate a dose during the lockout interval (76). Activation unit 10 may, for example, illuminate an LED or emit an audible tone. Activation unit 10 then proceeds to monitor for subsequent activation requests (66 of FIG. 7).

If the lockout interval has expired (yes branch of 74 of FIG. 8), activation unit 10 may determine whether servicing the user request would cause one or more thresholds to be exceeded depending on the nature of the activation request (75). Activation unit 10 may, for example, may determine whether delivery of an additional bolus would exceed a threshold number of dosages over a period of time. As another example, activation unit 10 may determine whether an adjustment to the flow rate may cause the flow rate to exceed a specified maximum flow rate, or fall below a minimum flow rate. Furthermore, activation unit may determine whether the amount of drug dispensed for each bolus may be incremented.

If servicing the user request would cause one or more threshold to be exceeded, activation unit 10 may indicate that the user request cannot be serviced, possibly by illuminating an LED or emitting an audible tone (94). Otherwise, activation unit 10 outputs an activation signal via link 8. In response to receipt of the activation signal, drug delivery system 4 dispenses a bolus to a location within the body of the patient. In other embodiments, drug delivery system 4 may increase a drug bolus amount of a pre-programmed drug schedule or adjust a flow rate.

Activation unit 10 may receive a response signal confirming the successful delivery of a drug bolus from drug delivery system 4, and may indicate the success to the user (82). Upon activation of drug delivery system 4 in response to the user request, activation unit 10 restarts the lockout interval, thereby reducing the risk of overdosage by the patient (72). If the activation was not successful, or if communication cannot be established with drug delivery system 4, activation unit 10 indicates the error to the user (84) and continues without restarting the lockout interval.

The preceding specific embodiments are illustrative of the practice of the invention. It is to be understood, therefore, that other expedients known to those skilled in the art or disclosed herein, may be employed without departing from the invention or the scope of the appended claims. For example, for exemplary purposes, many of these features of the invention have been described in reference to activation unit 10. The present invention, however, is not so limited. Rather, the use of a locking interval and other safety features may be readily incorporated directly into the drug delivery system.

All printed publications referenced hereinabove, including all patents and patent applications, are hereby incorporated by reference into the specification hereof, each in its respective entirety.

As those skilled in the art will appreciate readily upon reading the Summary of the Invention, the Detailed Description of the Preferred Embodiments and the claims set forth below, at least some of the devices and methods disclosed in the patents referenced herein may be modified advantageously in accordance with the teachings of the present invention.

In the claims, means-plus-functions clauses are intended to cover the recited structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

1. A method for activating an implantable drug delivery system, the method comprising:
maintaining a timer to time a lockout interval; and
rejecting a user request to activate an implantable drug delivery system prior to expiration of the lockout interval.

2. The method of claim 1, further comprising activating the implantable drug delivery system in response to a user request received after expiration of the lockout interval.

3. The method of claim 2, further comprising restarting the lockout interval upon activating the drug delivery system.

4. The method of claim 1, wherein maintaining a timer comprises setting the lockout interval in response to user input.

5. The method of claim 1, wherein maintaining a timer comprises maintaining a timer within an external activation device, the method further comprising:
receiving an input signal indicating the user request upon activation of a button of the external activation device; and
communicating an activation signal from the external activation device to the implantable drug delivery system in response to the input signal when the input signal is received after expiration of the lockout interval.

6. The method of claim 5, further comprises outputting an error indication from the external activation device when the input signal is received prior to the expiration of the lockout interval.

7. The method of claim 6, wherein outputting an error indication comprises illuminating a light emitting diode (LED) of the external activation device.

8. The method of claim 6, wherein outputting an error indication comprises outputting an audible tone from the external activation device.

9. The method of claim 1, wherein maintaining a time comprises maintaining a timer within the implantable drug delivery system, the method further comprising:
receiving an input signal indicating the user request upon activation of a button of an external activation device; and
communicating an activation signal from the external activation device to the implantable drug delivery system in response to the input signal.

10. The method of claim 9, wherein reject a user request comprises outputting an error signal from the implantable drug delivery system to the external activation device when the activation signal is received prior to expiration of the lockout interval.

11. The method of claim 10, further comprises outputting an error indication from the external activation device in response to the error signal.

12. The method of claim 11, wherein outputting an error indication comprises illuminating a light emitting diode (LED) of the external activation device.

13. The method of claim 11, wherein outputting an error indication comprises outputting an audible tone from the external activation device.

14. The method of claim 2, wherein activating the implantable drug delivery system comprises dispensing a bolus of a drug.

15. The method of claim 2, wherein activating the implantable drug delivery system comprises:
maintaining an bolus amount of the drug to dispense; and
adjusting the bolus amount in response to the user request.

16. The method of claim 2, wherein activating the implantable drug delivery system comprises adjusting a flow rate of the implantable drug delivery system in response to the user request.

17. A method for activating an implantable drug delivery system, the method comprising:
initiating a timer to time a lockout interval upon issuing a first dosage of a drug an implantable drug delivery system;
receiving an input signal indicating a user request for a second dosage of the drug from the implantable drug delivery system;
communicating an activation signal from an external activation device to the implantable drug delivery system in response to the input signal when the input signal is received after expiration of the lockout interval; and
rejecting the user request for the second dosage of the drug when the input signal is received prior to expiration of the lockout interval.

18. The method of claim 17, further comprising programmatically setting a lockout interval.

19. The method of claim 17, further comprising:
receiving a response communication from the implantable drug delivery system; and
restarting the timer when the response communication indicates the implantable drug delivery system issued the second dosage of the drug.

20. The method of claim 17, wherein rejecting the user request comprises outputting an error indication from the external activation device.

21. The method of claim 20, wherein outputting an error indication comprises illuminating a light emitting diode (LED) of the external activation device.

22. A computer-readable medium comprising instructions for causing a programmable processor to:
maintain a timer to time a lockout interval; and
reject a user request to activate an implantable drug delivery system prior to expiration of the lockout interval.

23. The medium of claim 22, wherein the instructions the programmable processor to maintain a software time.

24. The medium of claim 22, wherein the instructions the programmable processor to maintain a hardware time.

25. The medium of claim 22, wherein the instructions further cause the programmable processor to reset the lockout interval upon activating the drug delivery system.

26. The medium of claim 22, wherein the instructions further causing the programmable processor to set the lockout interval in response to user input.

27. The medium of claim 22, wherein the instructions cause the programmable processor to maintain a timer within an external activation device, and wherein the instructions further cause the programmable processor to:
receive an input signal indicating the user request upon activation of a button of the external activation device; and
communicate an activation signal from the external activation device to the implantable drug delivery system in response to the input signal when the input signal is received after expiration of the lockout interval.

28. The medium of claim 27, the instructions further causing the programmable processor to output an error indication from the external activation device when the input signal is received prior to the expiration of the lockout interval.

29. The medium of claim 22, wherein the instructions cause the programmable processor to maintain the timer within the implantable drug delivery systems, and wherein the instructions further cause the programmable processor to:
receive an activation signal from the external activation device; and
output an error signal from the implantable drug delivery system to the external activation device when the activation signal is received prior to expiration of the lockout interval

30. An apparatus comprising:
an input/output (I/O) device;
controller coupled to the I/O device to receive a user request to activate an implantable drug delivery system; and
a timer managed by the controller to time a lockout interval, wherein the controller outputs an activation signal to activate the implantable drug delivery system when the user request is received after expiration of the lockout interval.

31. The apparatus of claim 30, wherein the controller rejects the user request to activate the implantable drug delivery system when the user request is received prior to expiration of the lockout interval.

32. The apparatus of claim 30, wherein the controller outputs an error indication upon rejecting the request.

33. The apparatus of claim 30, further comprising a light emitting diode (LED), and wherein the controller illuminates the LED upon rejecting the request.

34. The apparatus of claim 30, wherein the controller restarts the time upon activating the drug delivery system.

35. The apparatus of claim 30, wherein the apparatus comprises an external activation device and further includes a telemetry unit to transmit the activation signal to the implantable drug delivery system.

36. The apparatus of claim 30, wherein the apparatus comprises the drug delivery system, and wherein the I/O device comprises a telemetry unit to receive a telemetric communication conveying the user request.

36. The apparatus of claim 30, wherein the timer comprises a software timer.

37. The apparatus of claim 30, wherein the timer comprises a hardware timer.

38. A system comprising:
an implantable drug delivery system; and
an activation unit operable by a user to request activation of the implantable drug delivery system.

39. The system of claim 38, wherein the activation unit includes a controller to reject a user request to activate the implantable drug delivery system prior to expiration of a lockout interval.

40. The system of claim 39, wherein the controller issues an activation signal to the implantable drug delivery system in response to a user request received after expiration of the lockout interval.

41. The system of claim 39, wherein the controller restarts the lockout interval upon activating the drug delivery system.

42. The system of claim 39, wherein the controller programmatically sets the lockout interval in response to user input.

43. The system of claim 39, wherein the controller outputs an error indication from the when the user request is received prior to the expiration of the lockout interval.

44. The system of claim 38, wherein the implantable drug delivery system dispenses a predetermined amount of drug upon activation, and wherein the implantable drug delivery system adjusts the bolus amount in response to the user request.

45. The system of claim 38, wherein the implantable drug delivery system adjusts a flow rate in response to the user request.

46. A system comprising:
means for initiating a timer to time a lockout interval upon issuance of a first dosage of a drug via an implantable drug delivery system;
means for receiving an input signal indicating a user request for a second dosage of the drug from the implantable drug delivery system;
means for communicating an activation signal from an external activation device to the implantable drug delivery system in response to the input signal when the input signal is received after expiration of the lockout interval; and
means for rejecting the user request for the second dosage of the drug when the input signal is received prior to expiration of the lockout interval.

47. The system of claim 46, further comprising means for programmatically setting a lockout interval.

48. The system of claim 46, further comprising:
means for receiving a response communication from the implantable drug delivery system; and
means for restarting the timer when the response communication indicates the implantable drug delivery system issued the second dosage of the drug.

49. The system of claim 46, further comprising means for outputting an error indication from the external activation device.

50. A method for activating an implantable drug delivery system, the method comprising:
receiving a user request to activate an implantable drug delivery system;
communicating an activation signal from an external activation device to the implantable drug delivery system in response to the user request; and
activating the implantable drug delivery system in response to the activation signal.

51. The method of claim 50, wherein activating the implantable drug delivery system comprises dispensing a bolus from the drug delivery system.

52. The method of claim 50, wherein activating the implantable drug delivery system comprises:
maintaining an amount of the drug to dispense for each bolus; and
adjusting the amount in response to the user request.

53. The method of claim 50, wherein activating the implantable drug delivery system comprises adjusting a flow rate of the implantable drug delivery system in response to the user request.
